(19) United States
(12) Patent Application Publication
(10) Pub. No.: US 2003/0066902 A1
(43) Pub. Date: Apr. 10, 2003

(54) Deodorant holder

(76) Inventor: Marianna Georgescu, St. Petersburg, FL (US)

Correspondence Address: John B. Dickman, III, Suite 1203, 2001 Jefferson Davis Highway, Arlington, VA, 22202, US

(21) Appl. No.: 09/957,592

(22) Filed: Sep. 21, 2001

(51) Int. Cl. A61L 9/04

(52) U.S. Cl. 239/57; 239/56; 239/53; 239/36

(57) ABSTRACT

An apertured container for holding perfume and perfume impregnated material comprising two halves which are easily opened and closed for placing fresh perfumed material therein and removing spent perfumed material therefrom, and locking means for holding said halves together. A suspension means is attached to the container, and can be used to attach the container to any desired spot. The container is in the form of a sporting device such as a baseball, football, soccer ball and the like.

### BACKGROUND OF THE INVENTION

This invention relates to hollow perfume and jewelry objects which contain perfume and perfume impregnated materials. The perfume and impregnated materials can be easily removed and replaced when other scents are desired and when the scents become exhausted. The scents escape through a plurality of apertures in the hollow objects. The hollow objects comprise two or more portions attached together and having locking means to hold them together when the object is in use.

### OBJECTS OF THE INVENTION

An object of the present invention is to provide an inexpensive perfume dispenser that can be attached to a body part or to clothing.

A further object of the invention is to provide an inexpensive perfume dispenser which can be used as a locket or other piece of jewelry.

A still further object of the invention is to provide a perfume dispenser which is easily opened and closed for insertion and removal of perfume impregnated material.

A still further object of the invention is to provide earrings, lockets and the like simulating common well-known objects such as footballs, bbaseballs, soccer and basketballs and the like.

A still further object is to provide an object as described which has two or more sections attached together and lockable to prevent accidental opening.

Other objects, features and advantages of the present invention will become apparent from the detailed description of the preferred embodiments of the invention which follows, when considered in light of the accompanying drawings, in which:

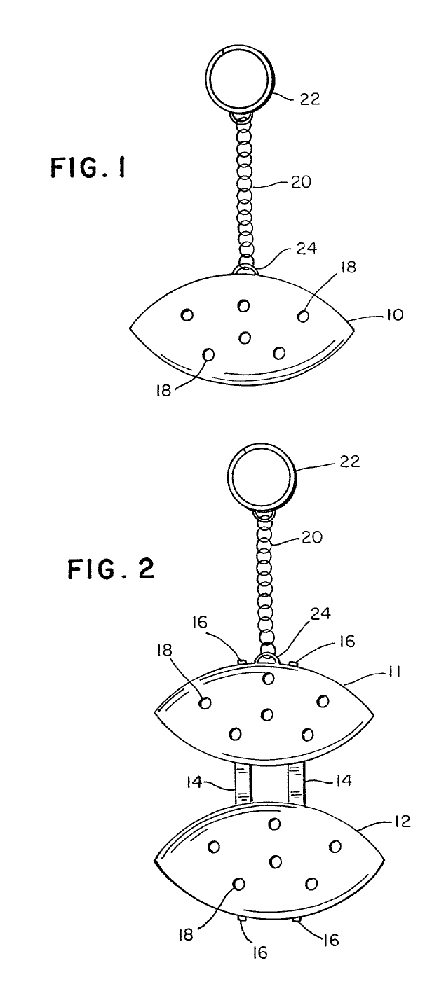

FIG. 1 is a plan view of one form of the device in closed position and showing the general appearance of a football.

FIG. 2 is a plan view of the device shown in FIG. 1 in it's open position read to receive a perfume impregnated material.

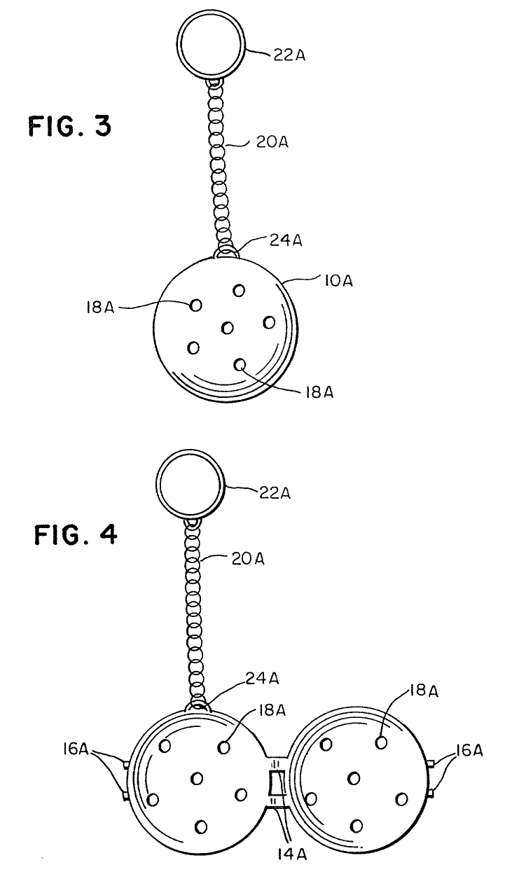

FIG. 3 is a plan view of another form of the invention in closed position and simulating the appearance of a round ball.

FIG. 4 is a plan view of the device shown in FIG. 3 in it's open position ready to receive a perfumed insert.

### DESCRIPTION OF THE PREFERRED EMBODIMENT

Referring now to FIGS. 1 and 2, a container 10 having two halves 11 and 12 attached together by means 14, said means being made of plastic, metal, cloth or any suitable material.

Mating engaging means 16, which can be pins, clamps, magnetic means or any other suitable device, hold the two or more sections 11 and 12 closed when desired. Apertures 18 are located at spaced intervals on the several parts and allow the perfume or other scents to escape into the atmosphere.

Suspention means 20, 22 and 24 comprise a chain 20, a loop 22, and a fastening means 24 attached to container 10, and allow the device to be suspended from the user's neck, ears, articles of clothing and other areas.

A perfume impregnated material, cartridge and the like, is placed inside the container 10 and the aromas then pass through the apertures 18 to scent the atmosphere.

In the embodiment shown in FIGS. 1 and 2, the container 10 stimulates a football.

Referring to FIGS. 3 and 4, similar to FIGS. 1 and 2, but simulating a baseball, there is shown a container 10a, comprising two halves 11a and 12a, having attaching means 14a of any suitable material and engaging means 16a to hold the two halves together when desired. In these FIGS. 3 and 4, the attaching means are shown offset to one side of the container 10a, but they maybe attached at any desired location.

Apertures 18a are located on both halves of container 10a.

20a, 22a and 24a show suspension means comprising a chain, a loop and a fastening means respectfully, the fastening means connected to container 10a to allow suspension of the container from any desired point.

In use, the container 10 or 10a is opened, perfume or perfume impregnated material (not shown) is placed in the open container, the container is then closed and fastened by the attaching means 16. The container 10 is then suspended from a persons ear, clothing or other selected area.

It is to be noted that the container 10 is shaped like a football, container 10a is shaped like a baseball. Any other balls, such as soccer, basketball, volley balls and the like,

Hockey pucks, baseball bats, hockey sticks and other sport paraphenalia are also considered to be within the purview of this invention.

In the claims:

1. A reusable perfume aroma dispensing container comprising a container having two halves, a plurality of apertures in said halves, said halves being attached to each other at any suitable spot and openable by a user to allow insertion of perfume and perfume containing material therein, and closable to hold said perfume and perfume containing material within said container, a locking means to hold said container closed when desired, and a suspention means for attaching said container to a desired spot.

2. A perfume dispensing device as set forth in claim 1 in which the container has a baseball shaped configuration.

3. A perfume dispensing container as in claim 1 in which the container has a football shaped configuration.

4. A perfume dispensing container as in claim 1 in which the container has a substantially shaped soccer ball configuration.

5. A perfume dispensing container as set forth in claim 1 in which said container has a substantially sporting device configuration.
